(19) United States
(12) Patent Application Publication
(10) Pub. No.: US 2014/0329741 A1
(43) Pub. Date: Nov. 6, 2014

(54) USE OF CYCLIC PEPTIDES FROM FLAXSEED FOR IMPROVING ANIMAL AND HUMAN HEALTH

(71) Applicant: University of Saskatchewan, Saskatoon (CA)

(72) Inventors: Bernard Laarveld, Saskatoon (CA); Andrew A. Olkowski, Saskatoon (CA)

(21) Appl. No.: 14/366,358

(22) PCT Filed: Dec. 19, 2012
(86) PCT No.: PCT/CA2012/001166

§ 371 Date: Jun. 18, 2014

Related U.S. Application Data
(60) Provisional application No. 61/577,217, filed on Dec. 19, 2011.

(51) Int. Cl. A61K 38/12; A61K 36/55; A23L 1/30

(52) U.S. Cl. CPC A61K 38/12; A23L 1/3002; A61K 36/55; A23V 2002/00
USPC 514/3.6; 514/21.1; 514/4.8

(57) ABSTRACT

The present application includes a method for improving human and animal health comprising administering an effective amount of a linus cyclopeptide (LCP) extract from flaxseed to a subject in need thereof. Also included are feeds, foodstuffs, functional food compositions, natural product compositions nutraceuticals and food additive compositions comprising an LCP extract from flaxseed.

The present application claims the benefit of priority of co-pending U.S. provisional patent application No. 61/577,217 filed on Dec. 19, 2011, the contents of which are incorporated herein by reference in their entirety.

### FIELD

The present application is directed to methods of using Linus cyclopeptide (LCP) extracts from flaxseed in food items or pharmaceutical compositions for improving the health of animals and humans.

### BACKGROUND

Flax (Linum usitatissimum, also known as common flax or linseed), a member of the family Linaceae, is cultivated for production of oil seeds and fiber, and is one of the oldest crops (Jhala, A. J. et al. Australian Journal of Basic and Applied Sciences, 2010, 4, 4304-4312; Manniche L (1999) An ancient Egyptian herbal. British Museum Press, London). The edible oil, linseed oil or flaxseed oil, is obtained by extraction of flaxseed. Consumption of flaxseed oil increases concentration of α-linolenic acid (ALA) in blood plasma (Barceló-Coblijn, G. et al. American Journal of Clinical Nutrition, 2008, 88, 801-809; Harper, C. R. et al. Journal of Nutrition, 2006, 136, 83-87; Kaul, N. et al. Journal of the American College of Nutrition, 2008, 27, 51-58). ALA is a precursor of the polyunsaturated fatty acids (PUFA), eicosapentaenoic acid (EPA) and docosahexaenoic acid (DHA) that reduce the risks of cardiovascular diseases (Barceó-Coblijn, G. ibid.; Harper, C. R., ibid; Gebauer, S. K. et al. American Journal of Clinical Nutrition, 2006, 83, S1526-1535S). Flaxseed contains secoisolariciresinol diglucoside (SDG), a phytochemical antioxidant that acts as a precursor of mammalian lignans and a phytoestrogen (Adolphe, J. L. et al. British Journal of Nutrition, 2010, 103, 929-938; Prasad, K. et al., International Journal of Angiology, 2004, 1, 7-14; Saggar, J. K.; et al. Nutrition and Cancer 2010, 62, (4), 533-542). Flaxseed also contains natural hydrophobic cyclic peptides comprising eight or nine amino acid residues. The term Linus cyclopeptide or LCP is used to refer to such cyclic peptides.

Many of the beneficial effects of flaxseed have been ascribed to the presence of omega-3 essential fatty acids, lignans and high fibre content in flaxseed. However certain biological effects have been reported for individual LCPs. For example, Linus cyclopeptide (LCP)-9 inhibits T-cell proliferation in response to concanavalin A. As a result of this discovery and the known role of T-cells in immunity (especially related to graft rejection and T-cell action) the following observations have also been reported in the literature: LCPs mitigate delayed hypersensitivity, skin allograft rejection and graft vs. host reactions. Conversely, LCPs inhibit interleukin-alpha and interleukin-2. LCPs also temper post-adjuvant polyarthritis and haemolytic immunity (Siemion, I. Z. et al. Archivum Immunologiae et Therapia Experimentalis, 1999, 47, 143-153).

The total concentration of LCPs in flaxseed is 0.2% making prior art methods of isolation of individual LCPs a laborious task (Gorski, A. et al. Transplantation Proceedings, 2001, 33, 553-553; Wieczorek, Z. et al. Peptide research 1991, 4, 275-283; Morita, H. et al. Tetrahedron 1999, 55, 967-976). Reaney and co-workers established improved methods for recovery of flaxseed oil LCPs by adsorption on silica gel and subsequent desorption using organic solvents of varying polarity (Reaney, M. J. et al. PCT Patent Application WO 2009/079792). Kaufman et al. (Kaufmann, H. P. et al. Chem. Ber.-Recl. 1959, 92, 2805-2809) describe a method of recovery of hydrophobic peptides from a precipitated “slime” obtained from flaxseed oil. Morita et al, (Matsumoto, T. et al. Phytochemistry 2001, 57, 251-260; Morita, H. et al. ibid; Morita, H. et al. Bioorganic and Medicinal Chemistry Letters, 1997, 7, 1269-1272; Matsumoto, T. et al. Tetrahedron 2002, 58, 5135-5140) describe the preparation of small amounts of hydrophobic peptides from flaxseed, press cake and roots. Stefanowicz (Acta Biochimica Polonica 2001, 48, 1125-1129) also disclosed a recovery of hydrophobic peptides from flaxseed, although this process does not allow for the separation and isolation of each hydrophobic peptide. A recent publication by Bruhl et al. (Journal of Agricultural and Food Chemistry, 2007, 55, 7864-7868) disclosed an analytical method for isolating small amounts of hydrophobic Linus cyclopeptides from flax oil.

### SUMMARY

Studies using a chicken model revealed that extracts of LCPs, when fed as part of the animal's diet, provided unexpected health benefits and therefore represent good candidates for applications related to improving animal and human health. For example, animals fed a diet comprising a mixture of LCPs were larger and had few incidents of a variety of health issues compared to controls. In particular, animals fed a diet comprising an LCP extract from flaxseed were found to have a lower incidence of enteritis, lower incidence of tissue and organ lesions and increased mitotic activity, compared to controls. Further LCP extracts from flaxseed were shown to have anti-cancer and antimicrobial (for e.g. antibacterial, antiviral and antifungal) activity.

The LCP extracts used in the methods and uses of the present application are those isolated from flaxseeds or flaxseed oil using methods known in the art for the separation and isolation of LCPs from flaxseed. In an embodiment, the LCP extract comprises all of the LCP's that are naturally present in flaxseed and each LCP is present in the ratio found in the natural source. It is also an embodiment, that the LCP extract comprises all of the LCP's that are naturally present in flaxseed wherein those LCPs that naturally contain methionine are present in LCP extract in their oxidized form (i.e. as methionine sulfoxide).

In an embodiment of the application, the LOP extract from flaxseed is obtained using a method comprising:

(a) combining flaxseed oil with an adsorbent that binds the hydrophobic peptides from the oil, followed by separation of the adsorbent and extraction of the hydrophobic peptides from the adsorbent using a suitable solvent; or
(b) extracting flaxseeds, or flaxseed oil, with a suitable solvent and separating the LCPs from the polar solvent using silica gel solid phase extraction using a suitable solvent.

The present application is directed to a method of improving the health of a subject comprising administering an effective amount of an LCP extract from flaxseed to a subject. The application also includes a use of an LCP extract from flaxseed to prepare a medicament, food stuff or nutraceutical to improve the health of a subject. Also included is an LCP extract from flaxseed for use to improve the health of a subject.

In one embodiment, the present application includes a method for improving the body weight gain of an animal comprising administering to the subject an effective amount of an LCP extract from flaxseed. Also included is a use of an LCP extract from flaxseed for increasing the body weight gain of a subject. Also included is an LCP extract from flaxseed for use to increase the body weight gain of a subject.

The present application includes a method for preventing or treating enteritis comprising administering an effective amount of an LCP extract from flaxseed to a subject in need thereof. Also included is a use of an LCP extract from flaxseed for preventing or treating enteritis. Also included is an extract from flaxseed for use in preventing or treating enteritis.

In yet another embodiment, the present application also includes a method for preventing or treating loss of intestinal villi comprising administering an effective amount of an LCP extract from flaxseed to a subject in need thereof. Also included is a use an LCP extract from flaxseed for preventing or treating loss of intestinal villi. Also included is an extract from flaxseed for preventing or treating loss of intestinal villi.

In still another embodiment, the present application also includes a method for preventing or treating organ and/or tissue lesions comprising administering an effective amount of an LCP extract from flaxseed to a subject in need thereof. Also included is a use of an LCP extract from flaxseed for preventing or treating organ and/or tissue lesions. Also included is an extract from flaxseed for preventing or treating organ and/or tissue lesions.

In still another embodiment, the present application also includes a method for enhancing cell cycle activity comprising administering an effective amount of an LCP extract from flaxseed to a subject in need thereof. Also included is a use of an LCP extract from flaxseed for enhancing cell cycle activity. Also included is an extract from flaxseed for use in enhancing cell cycle activity.

In still another embodiment of the present application the LCP extract from flaxseed is used as an antimicrobial agent. Therefore the present application also includes a method of inhibiting microbial growth or treating a microbial infection comprising administering an effective amount of an LCP extract from flaxseed to a subject or sample in need thereof. Also included is a use of an LCP extract from flaxseed to inhibit microbial growth or treat a microbial infection. Also included is an extract from flaxseed for inhibiting microbial growth or treating a microbial infection. In an embodiment, the microbe is selected from bacteria, viruses, fungi and yeasts.

In still another embodiment of the present application there is included a method of inhibiting fungal and/or yeast growth comprising administering an effective amount of an LCP extract from flaxseed to the fungus or yeast. Also included is a use of an LCP extract from flaxseed to inhibit fungal and/or yeast growth. Also included is an extract from flaxseed for inhibiting fungal and/or yeast growth.

In yet another embodiment, the present application includes a method of modulating gastro-intestinal tract microorganisms comprising administering an effective amount of an LCP extract from flaxseed to a subject in need thereof. Also included is a use of an LCP extract from flaxseed to modulate gastro-intestinal tract microorganisms. Also included is an extract from flaxseed for use to modulate gastro-intestinal tract microorganisms.

The present application includes a method for preventing or treating viral infections, such as inclusion body hepatitis, comprising administering an effective amount of an LCP extract from flaxseed to a subject in need thereof. Also included is a use of an LCP extract from flaxseed for preventing or treating viral infections. Also included is an extract from flaxseed for use in preventing or treating viral infections.

In an embodiment of the methods of the application, the administration of the effective amount of an LCP extract from flaxseed to a subject is by feeding a subject a diet that comprises an effective amount of an LCP extract from flaxseed. The application also includes a use of a diet that comprises an LCP extract from flaxseed to improve health of a subject. Also included is a diet that comprises an LCP extract from flaxseed for use to improve the health of a subject.

The present application also includes a feed or food stuff, a food supplement composition, a nutraceutical, a functional food composition, a natural product composition or a pharmaceutical composition, comprising an LCP extract from flaxseed.

Other features and advantages of the present disclosure will become apparent from the following detailed description. However, it should be understood that the detailed description and the specific examples, while indicating preferred embodiments of the disclosure, are given by way of illustration only because various changes and modifications within the spirit and scope of the disclosure will become apparent to those skilled in the art from this detailed description.

### BRIEF DESCRIPTION OF THE DRAWINGS

The application will now be described in greater detail with reference to the drawings in which:

FIG. 1 is a graph showing representative body weights (g) for broiler chickens fed basal diet (control group) and broiler chickens exposed to basal diet amended with an LCP extract (treatment group).

FIG. 2 shows pictures of representative examples of differences in inflammatory lesions observed in the intestines of broiler chickens from the control group fed basal diet (a) and broiler chickens exposed to a basal diet amended with an LCP extract (b).

FIG. 3 is a graph showing the comparison of bursa indices between broiler chickens fed basal diet (control group) and broiler chickens exposed to a basal diet amended with an LCP extract (treatment group).

FIG. 4 shows a comparison of histological features of bursa from control broiler chickens fed basal diet (a) and bursa from broiler chickens fed diets supplemented with an LCP extract (b). Original magnification; 100×.

FIG. 5 shows microscopic images from a section of intestines showing evidence of enteritis on gross examination in control broiler chickens fed a basal diet (a) and from broiler chickens fed diets supplemented with an LCP extract (b). Original magnification; 50×.

FIG. 6 shows examples of mitotic activity in intestinal mucosa of chickens. Black arrows indicate various stages of mitotic activity including prophase, metaphase, anaphase and telophase. FIG. 6a shows an example of more vigorous mitotic activity evidenced by larger numbers of mitotic figures in crypts which were typical of broiler chickens fed diets supplemented with an LCP extract (a) in comparison to control broiler chickens fed a basal diet (b). Original magnification; 400×.

FIG. 7 is a picture showing typical foot pad pathology seen commonly in commercial broiler chickens. Noteworthy is the evident soft tissue necrosis (arrow) affecting a large area of the foot, with hemorrhage and exposure of the deep tissues, as well as overall edema of the entire foot pad.

FIG. 8 contains pictures showing representative examples of foot lesions observed in broiler chickens from the control group fed a basal diet (a) and broiler chickens fed a basal diet amended with an LCP extract (b).

FIG. 9 contains pictures showing examples of a normal femoral head (a, arrow) and various degrees of degenerative changes of the coxo-femoral joint and femoral head commonly seen in broiler chickens (b, c, and d).

FIG. 10 shows a representative example of normal sporulated oocysts (a) and the oocysts' morphology in cultures treated with an LCP extract (b). Oocysts in the treated culture showed signs of severe injury as evidenced by blebbing of the oocyst shell. Some oocysts appeared to initially sporulate, but in comparison to viable sporulated oocysts, the spores appeared sickly, and were not viable. Oocyte injury, in particular blebbing, is consistent with the ionophore—like effects associated with cyclolinopeptides. Original magnification; 400×.

FIG. 11 shows the effect of an LCP extract on viability of spores isolated from chicken faecal matter. Original magnification; 400×.

FIG. 12 shows the effect of an LCP extract on C. albicans cells. Cells which were incubated only with DMSO (a) remained unstained (black arrows), and thus were viable. Several cells from cultures incubated with DMSO containing an LCP extract (b) were stained blue (upper two arrows in (b); both arrows in (c)), which indicated that these cells were not viable. Some cells showed significant injury as evidenced by blebbing of cellular membrane (c).

### DESCRIPTION OF VARIOUS EMBODIMENTS

### I. Definitions

Unless otherwise indicated, the definitions and embodiments described in this and other sections are intended to be applicable to all embodiments and aspects of the application herein described for which they are suitable as would be understood by a person skilled in the art.

As used in this application, the singular forms “a”, “an” and “the” include plural references unless the content clearly dictates otherwise. For example, an embodiment including “a compound” should be understood to present certain aspects with one compound, or two or more additional compounds.

In embodiments comprising an “additional” or “second” component, such as an additional or second compound, the second component as used herein is chemically different from the other components or first component. A “third” component is different from the other, first, and second components, and further enumerated or “additional” components are similarly different.

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps.

Terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

The term “subject” as used herein includes all members of the animal kingdom including birds, all aquatic species, and mammals.

The term “treating” or “treatment” as used herein and as is well understood in the art, means an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, diminishment of the reoccurrence of disease, and remission (whether partial or total), whether detectable or undetectable. “Treating” and “treatment” can also mean prolonging survival as compared to expected survival if not receiving treatment. “Treating” and “treatment” as used herein also include prophylactic treatment.

The term “feed”, “food stuff” or “feed composition” means any compound, preparation, mixture, or composition suitable for, or intended for intake by an animal subject, including humans, birds, and aquatic species.

The term “control” as used herein refers generally to a comparative treatment that is performed using identical conditions, except for a condition or parameter of interest.

The term “extract” as used herein refers to a concentrated fraction obtained by extraction of any variety and type of flaxseed and which exhibits the activity as described herein as verified by conducting one or more in vitro or in vivo biological evaluations. The extract can be used as such, if pharmaceutically acceptable, or the solvent of the resulting solution is removed and the residue used as such, or after further work-up, for example, further purification, and/or re-suspending in a suitable solvent.

A “functional food” as used herein is a food that is similar in appearance to, or may be, a conventional food that is consumed as part of a usual diet, and is demonstrated to have physiological benefits and/or reduce the risk of disease beyond basic nutritional functions, i.e. they contain an active ingredient.

A “nutraceutical” as used herein is a natural health product isolated or purified from foods that is generally sold in medicinal forms not usually associated with foods. A nutraceutical should have a physiological benefit or provide protection against disease.

### II. Methods/Uses of the Application

In a model trial using chickens, subjects fed a diet comprising an LCP extract from flaxseed had surprisingly overall improved health compared to subjects fed a control diet that did not comprise the LCP extract. In particular, the subjects receiving a diet comprising the LCP extract had improved growth rates and showed a decrease in morbidity and mortality compared to subjects receiving the control diet. Further, subjects receiving a diet comprising the LCP extract had a significant decrease in enteritis, foot lesions and femoral bone degeneration compared to subjects receiving the control diet. Still further, subjects receiving a diet comprising the LCP extract showed more vigorous mitosis in the bursa and in the intestinal mucosa compared to subjects receiving the control diet. Further in subjects receiving a diet comprising the LCP extract, there was an inhibition of infiltration of inflammatory cells into the intestinal walls which helped to prevent loss of villi in the intestines, compared to subjects receiving the control diet. Finally, in subjects receiving the control diet, the incidence of inclusion body hepatitis was 4-fold higher in comparison to subjects fed the LCP diet.

LCP extracts from flaxseed were also shown to be anti-coccidial and toxic to yeast sporulation and were shown to have fungicidal and antibiotic activity.

Accordingly, the present application relates to methods for improving health comprising administering an effective amount of an LCP extract from flaxseed to a subject in need thereof. The application also includes a use of an LCP extract from flaxseed to prepare a medicament, food stuff or a nutraceutical to improve health of a subject. Also included is an LCP extract from flaxseed for use to improve the health of a subject.

The present application also relates to methods for improving health comprising feeding a subject a diet that comprises an effective amount of an LCP extract from flaxseed. The application also includes a use of a diet that comprises an LCP extract from flaxseed to improve health of a subject.

Also included is a diet that comprises an LCP extract from flaxseed for use to improve the health of a subject.

In embodiments of the application, to improve health comprises one or more of: increasing growth rates and/or body weight, decreasing morbidity, decreasing mortality, decreasing enteritis, decreasing fungal growth, modulating gastro-intestinal tract micro-organisms, decreasing viral growth, enhancing cell-cycle activity and inhibiting infiltration of inflammatory cells into the intestinal mucosa.

In an embodiment, the increasing, decreasing or inhibiting of various functions by the LCP extract from flaxseed is any increase, decrease or inhibition of said function compared to a control, for example a control diet. The control diet is a diet that is identical to the diet comprising an LCP extract from flaxseed, with the exception that it does not comprise the LCP extract from flaxseed.

In an embodiment, the increase in growth rate is assayed by measuring average body weight over time. In the chicken model, chickens fed a basal diet which included an LCP extract from flaxseed showed significantly greater average body weights at 21 days and 35 days after being fed this diet. Accordingly, the present application also includes a method for increasing the body weight of a subject comprising feeding the subject a diet that comprises an effective amount of an LCP extract from flaxseed. Also included is a use of a diet that comprises an LCP extract from flaxseed for increasing the body weight of a subject. In an embodiment, the increase in the body weight of the subject is any increase compared to subjects fed a control diet that does not contain the LCP extract from flaxseed.

The incidence of enteritis, or inflammation of the intestine, in the chicken model study was significantly lower in the chickens fed a basal diet which included an LCP extract from flaxseed compared to the control group. Further, the severity of the lesions in the group receiving the diet comprising the LCP extract was also much lower compared to the control group. Accordingly, the present application also includes a method for preventing or treating enteritis comprising administering an effective amount of an LCP extract from flaxseed to a subject in need thereof. Also included is a use of an LCP extract from flaxseed for preventing or treating enteritis. The present application also includes a method for preventing or treating enteritis comprising feeding a subject a diet that comprises an effective amount of an LCP extract from flaxseed. Also included is a use of a diet that comprises an LCP extract from flaxseed for preventing or treating enteritis.

Still further, in subjects receiving the diet comprising an LCP extract from flaxseed, there was an inhibition of infiltration of inflammatory cells in the intestinal walls which helped to prevent loss of villi in the intestines, compared to subjects receiving the control diet. Accordingly, the present application also includes a method for preventing or treating loss of intestinal villi comprising administering an effective amount of an LCP extract from flaxseed to a subject in need thereof. Also included is a use of an LCP extract from flaxseed for preventing or treating loss of intestinal villi. The present application also includes a method for preventing or treating loss of intestinal villi comprising feeding a subject a diet that comprises an effective amount of an LCP extract from flaxseed. Also included is a use of a diet that comprises an LCP extract from flaxseed for preventing or treating loss of intestinal villi.

Reducing loss of intestinal villi aids in maintaining and/or restoring normal physiological function and health of the intestinal mucosa. The results observed in the present chicken study suggest that subjects fed a diet supplemented with an LCP extract from flaxseed will have a higher potential for re-generation of damaged villi. This is of value in the treatment of enteritis, where regeneration of damaged intestinal mucosa would be a very desirable feature. Continuous, active proliferation of the enterocytes at the crypt level is beneficial for the physiology of the entire villus and any interruption in that process will inevitably lead to the death of the villus.

The incidence of foot lesions and femoral bone degeneration in the chicken model study was lower in the chickens fed a basal diet which included an LCP extract from flaxseed compared to the control group. Accordingly, the present application also includes a method for preventing or treating organ and/or tissue lesions comprising administering an effective amount of an LCP extract from flaxseed to a subject in need thereof. Also included is a use of an LCP extract from flaxseed for preventing or treating organ and/or tissue lesions. The present application also includes a method for preventing or treating organ and/or tissue lesions comprising feeding a subject a diet that comprises an effective amount of an LCP extract from flaxseed. Also included is a use of a diet that comprises an LCP extract from flaxseed for preventing or treating organ and/or tissue lesions.

Still further, subjects receiving the feed comprising an LCP extract from flaxseed showed a pro-mitotic effect in the bursa, intestinal mucosa and in the enterocytes compared to subjects receiving the control diet. Accordingly, the present application also includes a method for enhancing cell-cycle activity comprising administering an effective amount of an LCP extract from flaxseed to a subject in need thereof. Also included is a use of an LCP extract from flaxseed for enhancing cell-cycle activity. The present application also includes a method for enhancing cell-cycle activity comprising feeding a subject a diet that comprises an effective amount of an LCP extract from flaxseed. Also included is a use of a diet that comprises an LCP extract from flaxseed for enhancing cell-cycle activity. In an embodiment, the enhancement in cell-cycle activity is any enhancement compared to subjects fed a control, such as a control diet that does not contain the LCP extract from flaxseed. In another embodiment, the enhancement in cell-cycle activity is in the bursa, intestinal mucosa and/or in the enterocytes. In an embodiment, enhancing cell-cyclic activity is via stimulation of mitosis and leads to more vigorous regeneration of enterocytes in the crypt (which may be beneficial for regeneration of intestinal villi).

In still another embodiment of the present application the LCP extract from flaxseed is used as an antimicrobial agent. Therefore the present application also includes a method of inhibiting microbial growth or treating a microbial infection comprising administering an effective amount of an LCP extract from flaxseed to a subject or sample in need thereof. Also included is a use of an LCP extract from flaxseed to inhibit microbial growth or treat a microbial infection. Also included is an extract from flaxseed for inhibiting microbial growth or treating a microbial infection. In an embodiment, the microbe is selected from bacteria, viruses, fungi and yeasts.

In still another embodiment of the present application, there is included a method of inhibiting fungal growth comprising administering an LCP extract from flaxseed to the fungus. Also included is a use of an LCP extract from flaxseed to inhibit fungal growth. The inhibition of the fungal growth may be in a subject and, therefore, is effected by administering an effective amount of the LCP extract to the subject. In an embodiment, the fungus is a yeast.

In yet another embodiment, the present application includes a method of modulating gastro-intestinal tract microorganisms comprising administering an effective amount of an LCP extract from flaxseed to a subject in need thereof. Also included is a use of an LCP extract from flaxseed to modulate gastro-intestinal tract microorganisms. By “modulating gastro-intestinal tract microorganisms” it is meant that the LCP extract from flaxseed regulates the growth of pathological and beneficial microbial strains for an improved balance of beneficial microbes leading to overall improved gastrointestinal health.

The present application includes a method for preventing or treating viral infections, such as inclusion body hepatitis, comprising administering an effective amount of an LCP extract from flaxseed to a subject in need thereof. Also included is a use of an LCP extract from flaxseed for preventing or treating viral infections. Also included is an extract from flaxseed for use in preventing or treating viral infections.

In another embodiment of the application, the LCP extract from flaxseed is a mixture of LCPs obtained by extraction of flaxseed oil using any known method. Flaxseed oil is obtained by mechanical or solvent extraction. Mechanical methods include, for example, pressing, grinding, emulsifying, extruding, blending, and/or exposure to ultrasound waves, microwaves and infrared radiation. In an embodiment, mechanical extraction includes methods of continuous expeller pressing. Seeds may be ground, flaked, extruded, and heated prior to oil expeller press extraction. Continuous expeller pressing may be conducted in a single press or in multiple presses. The oil may be recovered from the seed meal using a continuous extruder with an oil extraction cage. Oil may also be extracted from seed using low polarity organic solvents such as hexane and ethyl acetate, in addition to intermediate polarity organic solvents such as ethanol and butanol. The peptides and oil could be recovered from aqueous streams in the form of free oil or oleosomes. Those skilled in the art would be familiar with means of extraction of oil.

In an embodiment of the application, the LCP extract is obtained by combining flaxseed oil, which is commercially available or is obtained from flaxseeds using known methods as described above, with an adsorbent that binds the hydrophobic peptides from the oil, followed by separation of the adsorbent and extraction of the hydrophobic peptides from the adsorbent using suitable solvents, such as ethyl acetate or 10% methanol in chloroform. Such a method is described in Reaney et al. PCT Patent Application No. WO 2009/079792, the relevant contents of which are incorporated herein by reference. In another embodiment of the application, the LCP extract from flaxseeds is obtained from flaxseed using a method described in Gui et al. J. Agric. Food Chem. 2012, 60:8571-8579 or Gui et al. J. Agric. Food Chem. 2012, 60:8580-8589, the relevant contents of which are incorporated herein by reference. Accordingly, flaxseeds are ground and dried, or flaxseed oil is expressed from flaxseed, and either are extracted with a polar solvent such as acetone, ethyl acetate or 10% methanol in chloroform, for example using a thimble extractor. Silica gel solid phase extraction is subsequently used to separate the LCPs from other polar compounds, such as pigments, phospholipids and waxes. LCP's are known to elute from silica gel using ethyl acetate or 10% methanol in chloroform or another solvent or mixture of solvents having a similar polarity. Again, LCP-rich fractions are combined, the solvent removed and are used as the LCP extract of the present application.

Freshly pressed flaxseed oil comprises nine LCPs, named as LCP-1, LCP-2, LCP-3, LCP-4, LCP-5, LCP-6, LCP-7, LCP-8 and LCP-9, therefore it is an embodiment that the LCP extract from flaxseed oil comprises, consists essentially of or consists of all of these peptides in amounts that correspond to their amounts found in the flaxseed. The structures of each of the peptides has been reported (see Gui et al. J. Agric. Food Chem. 2012, 60:8571-8579). Note that LCP-3 corresponds to the oxidized form of LCP-2 (methionine oxidized to methionine sulfoxide) and LCP-7 corresponds to the fully oxidized form of LCP-8 and LCP-9. Since the methionine sulfur atom is susceptible to oxidation in air upon standing, it is possible that the LCP extract will not contain any of LCP-2, LCP-8 and/or LCP-9. Accordingly, it is an embodiment of the application that the LCP extract from flaxseed comprises, consists essentially of or consists of LCP-1, LCP-3, LCP-4, LCP-5, LCP-6 and LCP-7. In a further embodiment, the ratio of each of LCP-1, LCP-3, LCP-4, LCP-5, LCP-6 and LCP-7 in the extract corresponds to the composition ratio of these LCPs found in the corresponding flaxseed. The LCP compositions field grown seed from various flax cultivars is reported in Gui et al. J. Agric. Food Chem. 2012, 60:8571-8579) and the LCP compositions in flaxseed fractions (cotyledon, seed coat and whole seed) and after different processing procedures is reported in (see Gui et al. J. Agric. Food Chem. 2012, 60:8580-8589). It is an embodiment that the LCP extract from flaxseed comprises, consists essentially of or consists of:

(a) about 15% to about 30% of each of LCP-1, LCP-3 and LCP-5,
(b) about 10% to about 20% of LCP-7; and
(c) about 2% to about 15% of each of LCP-4 and LCP-6.

In a further embodiment of the application the LCP extract from flaxseed comprises greater than about 75, 80, 85, 90 or 95% of LCPs from flaxseed.

The subject that is fed a diet comprising the LCP extract from flaxseed in the methods and uses of the present application is any subject, including human, that would benefit from such a diet. In an embodiment, the subject is an animal selected from livestock and pets. In a further embodiment the animal is selected from a bovine, feline, equine, porcine, canine, aquatic, avian, caprine, ovine, and murine animal. In another further embodiment, the bovine animal is selected from beef cattle and dairy cows; the porcine animal is a hog; the ovine animal is a sheep; the avian animal is a poultry animal, such as chickens, ducks or turkeys; the caprine animal is a goat; the aquatic animal is selected from fish, crayfish and crustaceans; the canine animal is a domesticated dog; and the feline animal is a domesticated cat. The animals can, for instance, be adults, adolescents or infants. In yet another embodiment, the animal is an avian selected from chickens, turkeys and ducks, for example, chickens.

The treatment methods of the present application comprise administering to a subject a diet comprising an effective amount of an LCP extract from flaxseed. By “administering to a subject a diet” it is meant that the LCP extract from flaxseed is fed to the subject. In one embodiment, the LCP extract is simply added to the subject's basic or basal diet. In an alternative embodiment, the LCP extract is in a separate supplement given to the subject as an oral, or other, dosage form. The basic or basal diet, and the oral dosage forms are, for example, in solid or liquid form. Preparation of dosage forms comprising the LCP extract from flaxseed for administering to a subject can be done using methods known in the art.

In certain embodiments, the diet of the subjects being treated will be supplemented with the LCP extract from flaxseed as required and/or desired. This can be accomplished, for example, by providing the subject with feeds comprising the LCP extract from flaxseed. This feed comprising the LCP extract can be provided once, twice, or more times per day. In an embodiment, a single feeding per day of feed comprising the LCP extract, e.g. in the morning, is administered. In another embodiment, feed of the subjects is supplemented with a dry mixture or coating of the LCP extract from flaxseed to provide the beneficial compounds in the desired amounts. Alternatively, as mentioned above, in another embodiment, the LCP extract from flaxseed is fed in a bolus, capsule, pill, parenteral, gastric tube or other unit dose form known to a person skilled in the art. When feeding the LCP extract from flaxseed in the subjects' daily rations, the LCP extract can be blended with the feed rations in conventional hoppers or mixers from which the feed is dispensed. In this regard, a daily supply of feed can be blended with the LCP extract, or sufficient blended feedstuff containing the LCP extract can be prepared to feed the subjects over a number of days or weeks. These and other variations in the feeding or other administration aspects will be apparent to skilled artisans.

As used herein, the term “effective amount” or “therapeutically effective amount” means an amount effective, at dosages and for periods of time necessary to achieve the desired result. Effective amounts may vary according to factors such as the health condition, age, sex and/or weight of the subject. The amount of the LCP extract that will correspond to such an amount will vary depending upon various factors, such as the formulation, the route of administration, the type of disease or disorder (if any), the identity of the subject or host being treated, and the like, but can nevertheless be routinely determined by one skilled in the art. In an embodiment of the application, the effective amount of the LCP extract from flaxseed is about 0.1 g/kg to about 10 g/kg or about 0.5 g/kg to about 5 g/kg.

### III. Feeds of the Application

The present application also includes a feed or food stuff, a food supplement composition, a nutraceutical, a functional food composition, a natural product composition or a pharmaceutical composition, comprising an LCP extract from flaxseed.

For the feed or food stuff, the type and components of the feed are not particularly limited as long as the LCPs in the LCP extract from flaxseed are not degraded or otherwise affected by these components. In general, the LCP extract can be added to feeds and food stuffs such as feeds and food stocks for humans, livestock animals, pet foods, and other supplements for animals. An animal feed as used herein is any composition for animal nutrition, in solid or in liquid form. Feed is the main source of energy and nutrition for animals and is usually of animal or plant origin. Thus, feed may be defined as a substance with sufficient nutritional value to allow for growth and maintenance of adequate body conditions of an animal. In a typical embodiment, an animal feed composition consists of pellets, meal, grains, extruded or expanded grains, tablets, powder or bolus forms. For example, a feed composition comprises dry forages and roughages, energy feeds, protein feeds, mineral feeds, vitamin feeds, yeast products, normal premix, cornmeal, cotton seed, wheat gluten, maize silage, rutabaga, sugar beet pulp, apple pulp, ryegrass, fescue grass, alfalfa feed concentrate and/or feed supplement. In one embodiment, the feed or feed additive is in a form, and/or the composition is, approved by a governmental institution such as the FDA, the U.S. Department of Agriculture or the Canadian Food Inspection Agency. In Europe, the Task Force on Animal Feeding of the Codex Alimentarius Commission (CAC) as well as the Animal Welfare Act (AWA) provides definitions of animal feed. In another embodiment, the application includes an animal feed within the definition of “animal feed” in section 201(x) of the FFDCA supplemented with an LCP extract from flaxseed.

The feed and food stuffs of the present invention can be manufactured by adding the LCP extract from flaxseed to a component of a feed or a food stock. The concentration of the LCP extract contained in the feed and food stock is not particularly limited and may be appropriately adjusted depending on the subject's species, body weight, age, sex, intended use, health condition, feed component, etc., and is generally in an amount from about 0.01% (w/w) to about 10% (w/w), about 0.1% (w/w) to about 5% (w/w), or about 0.5% (w/w) to about 2% (w/w), of the total amount of the feed based on a dry form.

The LCP extract from flaxseed may be added to and mixed in a feed or food stock component without modification, but in the case of addition and mixing of a powdery or solid substance to the feed or food stock, the LCP extract may also be modified before use into a liquid or a gel for the purpose of facilitating the mixing in a feed or food stock. In this case, water; a vegetable oil such as soybean oil, rapeseed oil, or corn oil; a liquid animal oil; and/or a water-soluble polymer compound such as polyvinylalcohol, polyvinylpyrrolidone, or polyacrylic acid, may be used as a liquid carrier.

The LCP extract from flaxseed may also be formulated into a food supplement composition, a nutraceutical composition, a functional food composition or a natural product composition that is suitable for administration to subjects, including humans and other animals (for example birds and aquatic species). The compositions can be administered in various forms, such as, but not limited to a powder, a bulb, a capsule, a pill, a tablet, a liquid, a gel, a suspension, a cream or an ointment. They can be administrated, for example, orally, rectally, parenterally, intravaginally or transdermally or via a bolus administration such as via alimentary bolus administration. The food supplement compositions, nutraceutical compositions, functional food compositions or natural product compositions will comprise the LCP extract from flaxseed and at least one ingestible carrier.

The following non-limiting examples are illustrative of the present application:

### EXAMPLES

### Example 1

### Extraction of CLP's from Flaxseed

The mixture of cyclic peptides for the present in vivo and in vitro experiments was obtained from commercially available flaxseed oil using known methods of extraction. Therefore, flax oil was mixed with a suitable adsorbent having strong affinity for hydrophobic peptides from the oil. Note: several adsorbents with different chemistry are commercially available, and can be used for this purpose. In the present example, the adsorbent was silica. The absorbed peptides were then eluted from the adsorbent medium using a polar solvent, for example a solvent with a polarity similar to ethyl acetate or 10% ethanol in chloroform. Note: In addition to these solvents, many other solvents and their combinations with similar polarity characteristics can be used for this purpose. The solvent was removed and the residue dried.

### Example 2

### Linus Cyclopeptide-Amended Diets

One hundred broiler chicks (7 days old) were given feed amended with an LCP extract from flaxseed. The poultry feed was purchased from Federated Co-Operatives Limited. The LCP extract was homogenized and was then incorporated into the feed at a concentration of 0.2% (w/w) of total peptide and was used throughout the feeding trials.

The in vivo trial was conducted using the chicken as a model subject. From a pool of 200 day old commercial broiler chicks, 50 chicks were randomly allocated to a control group and 100 chicks were randomly allocated to a treatment group. Basal diet was un-medicated commercial broiler chicken diet formulated to fulfill the nutritional requirements of broiler chickens. The control group was offered the basal diet, whereas the treatment group was offered the basal diet amended with an LCP extract at an amount of 2 g per kg. Feed and water were provided ad libitum.

A standard experimental protocol included daily monitoring for potential adverse effects with emphasis on signs such as feed refusal, decreased appetite, somnolence, fatigue, exercise intolerance, increased respiratory rate, cyanosis, and gastrointestinal disorder. Birds, randomly selected from control and treatment groups, were euthanized weekly (for the first 4 weeks) and at the termination of the experiment in order to evaluate the effects of the LCP extract on internal organs. A physical examination was performed prior to data collection. Detailed gross post-mortem examination was performed on all mortalities and euthanized birds, and representative samples of organs were collected for microscopic and biochemical evaluation. The experiment was terminated at day 84 of chronic exposure for logistic reasons.

### Major Findings and Observations

### (a) Behaviour, General Physiology, Growth Parameters, and Overt Clinical Evaluation

Trial 1.

The amendment of basal diet with an LCP extract at a level of 2 g per diet did not cause feed refusal and had no effect on water consumption. All birds appeared physiologically normal and showed normal appetite. Growth rate, as measured by body weight during the acute phase of the study (first 3 weeks of exposure), was comparable with the control group fed the basal diet (Table 1).

At any point, over the 84 days of observations, there was no evidence of any adverse effects associated with the administration of the LCP extract nor did the subjects consider the feed unpalatable.

During the course of this experiment several observations were made indicating that the chickens fed the diet comprising the LCP extract from flaxseed actually appeared more robust in comparison to the controls. Although there were no significant differences in body weight gain between the control group and group fed diet fortified with the LCP extract from flaxseed, it is noteworthy that there was a trend indicative that chickens fed the diet supplemented with the LCP extract from flaxseed showed slightly higher gain than the control subjects, and this trend was observed consistently at each point of measurement (Table 1). This observation prompted the decision to conduct an additional trial.

Trial 2.

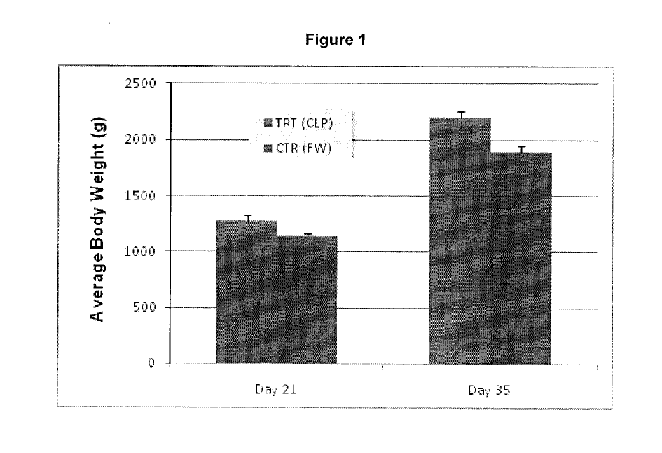

The second in vivo trial was an acute study with exposure time for 35 days, where the dose of the LCP extract from flaxseed in the diet was set at 4 g/kg. All chickens were fed ad libitum. There was no mortality or morbidity. During the course of this trial all birds appeared physiologically normal, so in essence, this trial confirmed all observations made during the first trial. However, the growth rate of chickens fed the diet comprising the LCP extract was significantly higher (p<0.01) in comparison to the control group fed the basal diet (FIG. 1).

### (b) Mortality and Morbidity

The standard experimental protocol included daily monitoring of morbidities and mortalities. During the course of Trial 1 (84 days), 4 broiler chickens were euthanized and 3 birds died. The reason for euthanasia was leg problems. In all cases the cause of death was associated with inclusion body hepatitis. Table 2 shows the distribution of mortalities and morbidities.

In the first Trial, both morbidity and mortality were lower in the treatment group in comparison to the control group. There was no mortality or morbidity during Trial 2.

### (c) Detailed Clinical and Gross Post Mortem Evaluation

Randomly selected birds from control and treatment groups were euthanized weekly (for the first 4 weeks) and at the termination of the experiment in order to evaluate the effects of the LCP extract from flaxseed on internal organs.

On detailed clinical examination prior to euthanasia, all birds from both control and treatment groups appeared clinically normal during the weekly sampling periods (first 4 weeks). At the terminal sampling (day 84), one chicken from the control group showed post mortem evidence of ascites associated with chronic congestive heart failure.

The dietary administration of the LCP extract from flaxseed did not cause any gross pathological changes in brain, liver, kidney, skeletal muscle, heart, gastrointestinal tract, and skin. Some grossly visible inflammatory lesions were apparent in the intestinal mucosa and serosa in specimens from both control and treatment groups (Table 3).

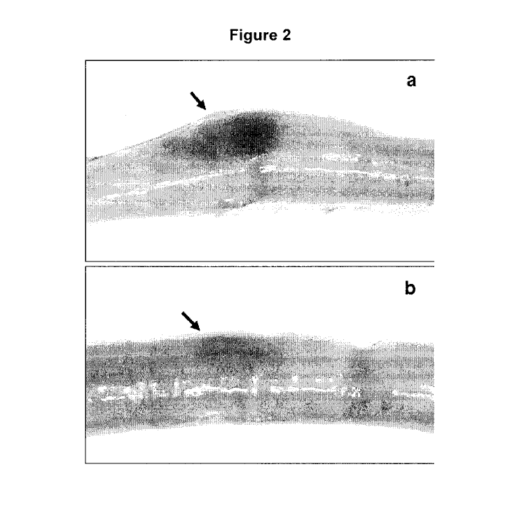

Inflammatory lesions are very commonly seen in many commercial broiler chickens, and the incidence of these lesions in the control group was within the range documented in the inventors' lab. However, in the context of the present study, it is noteworthy that the frequency of occurrence of the lesions was considerably lower in the treatment group in comparison to the control group. Interestingly, in addition to lower incidence, the severity of lesions was considerably lower in broiler chickens fed a diet amended with the LCP extract from flaxseed (FIG. 2).

### (d) Morphometric Evaluation

Because of the evidence suggesting that dietary supplementation with an LCP extract of flaxseed may have properties reducing inflammatory responses (see previous section, above) a detailed examination of specific organs important in the development of immune competence was carried out. One focus was on the bursa (of Fabricius), which, in birds, is a specialized organ involved in the development of immune cells. For the purpose of this study, the whole bursa was excised, blot dried, and weighed. For morphometric comparison, bursa weight was expressed as index of body weight (g/100 g BW).

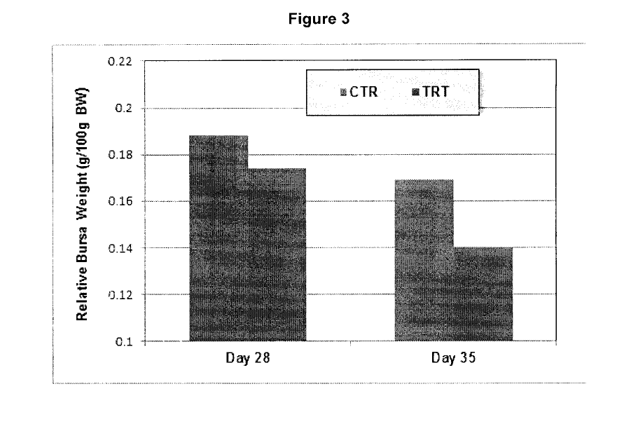

Interestingly, broiler chickens fed diets amended with an LCP extract from flaxseed had relatively smaller bursa than those fed control diets (FIG. 3).

Observations of a lower incidence of enteritis and a reduction in magnitude of inflammatory processes in broiler chickens fed a diet supplemented with an LCP extract of flaxseed is of great interest, as this action of an LCP extract would be very desirable for many reasons. For instance, the observed effect could be associated with anti-inflammatory properties, or could be associated with mediation of factors causing inflammatory responses (e.g. bacteria).

While not wishing to be limited by theory, the fact that supplementation of the diet with an LCP extract of flaxseed resulted in reduced bursa size may be interpreted that some immune-suppressive action of an LCP extract from flaxseed is possible. However, such immune-suppressive properties of the LCP extract must be viewed as patho-physiologically beneficial, because the observed reduction in bursa size was not associated with increased susceptibility to pathogens.

It is commonly accepted that the decreased size of bursa is associated with decreased immunity, and thus higher susceptibility to viral and bacterial infections. Although at first, the situation where the reduction in the bursa size did not result in increased susceptibility to infectious agents would appear paradoxical, further microscopic evaluation of bursa provided some explanation. Details are discussed in the ensuing paragraphs.

### (e) Microscopic Evaluation

At each sampling interval, sections of liver, intestines (normal and affected sections), skin, and bursa were collected for light microscopic evaluation. Samples of tissue were taken from similar anatomical areas representative of control birds and treated birds. The specimens were preserved in phosphate-buffered formaldehyde. Following the fixation, sections were embedded in paraffin wax, processed for routine light microscopy, and stained with haematoxylin and eosin. There were no indications of any histo-pathological changes in the liver, normal intestinal tissue, skin, or bursa.

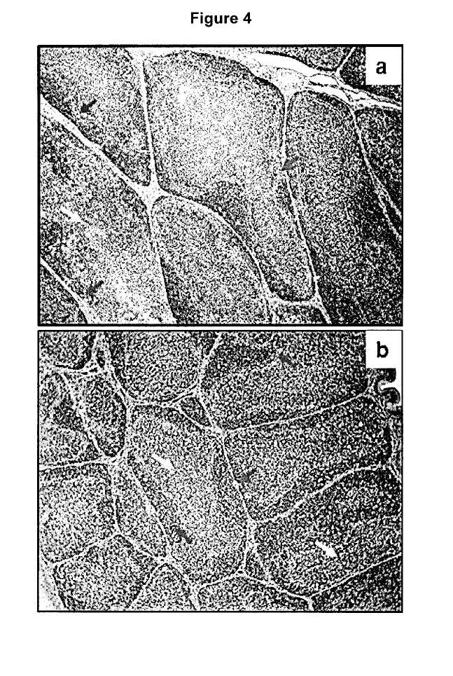

The bursa from control broiler chickens fed the basal diet showed some qualitative histological difference when compared to the bursa of broiler chickens fed diets supplemented with an LCP extract from flaxseed (FIG. 4).

Although both specimens show normal histology of cortex and medulla, there are apparent qualitative differences. In particular, noteworthy are considerable differences in numbers of cells populating medullary regions in bursa from control broiler chickens (FIG. 4a, white arrows) in comparison to similar areas of bursa from broiler chickens fed diets supplemented with an LCP extract (FIG. 4b white arrows). A similar difference in cellular density features are also apparent in cortical areas (indicated by darker arrows).

Furthermore, closer examination of the morphological characteristics revealed that cells from the control broiler chickens' specimens were poorly stained (a sign of poorer health), and many showed evidence of apoptosis, characterized by cytoplasmic vacuolization, eosinophilia, and nuclear pyknosis. Overall, cells in the bursa from broiler chickens fed diets supplemented with an LCP extract from flaxseed appeared well stained (a sign of health), and many showed signs of vigorous mitosis.

While not wishing to be limited by theory, if the observed features, such as the histologically evident higher density of cellular elements in the bursa of treated broiler chickens, are interpreted in the context of the lower bursa weight observed in this group, it can be inferred that, the relative weight of bursa decreased, but this decrease in size was compensated by increases in cell population, and as assessed by morphological features, better cell health. This reasoning seems plausible, and this is consistent with the observation that, despite this apparent decline in bursa size, the broiler chickens fed a diet supplemented with an LCP extract from flaxseed did not show any signs of impaired immunity.

### (f) Study of the Affected Intestinal Tissue:

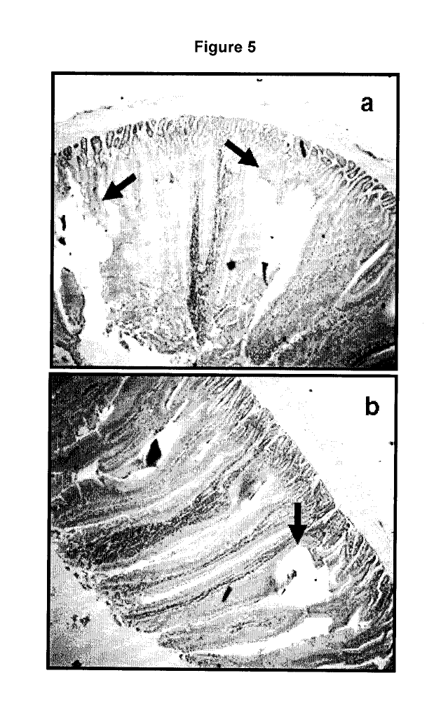

There were no indications of any histo-pathological changes in intestinal tissue that appeared normal on gross examination. However, microscopic examination of areas affected by inflammatory processes revealed significant histo-pathological changes, and there were differences in the severity of lesions between control and treated groups (FIG. 5).

In both cases, there is considerable disruption of intestinal mucosa architecture, with evident loss of villi. In some areas, there is significant hyperemia and congestion. However, noteworthy is the considerably higher grade of damage to the mucosa in the affected intestines from control broiler chickens fed basal diet (FIG. 5a, arrows) in comparison to broiler chickens fed diets supplemented with an LCP extract from flaxseed (FIG. 5b, arrow).

As discussed above, in addition to the higher incidence of enteritis in control broiler chickens in comparison to birds fed a diet supplemented with an LCP extract from flaxseed, it was also observed that the lesions in the treated group were less severe. The microscopic evaluation of the lesions confirmed this. However, closer examination of affected areas showed hyperemia, and also revealed massive infiltration of lamina propria of the affected villi with macrophages, lymphocytes, and granulocytes. Considering that the cells infiltrating intestinal mucosa are immune cells constituting the first line of defense mechanisms, these features are consistent with inflammatory responses mounted by the host to fend off invasion of intestinal pathogens. Therefore, observations of a lower incidence of enteritis, as well as reduction in the magnitude of inflammatory processes that occurred in broilers fed a diet supplemented with an LCP extract from flaxseed, taken together suggest that the LCP extract may have: 1) aided in mounting more effective defenses, 2) controlled negative aspects in inflammatory responses, and in essence acted as a selective anti-inflammatory agent, and 3) controlled factors causing inflammatory responses (e.g. bacteria, viruses, or parasites). While not wishing to be limited by theory, it is more likely that the putative beneficial action of the LCP extract from flaxseed observed in this trial may have resulted from a combination of the above-listed possibilities.

### (g) Important Aspects of Intestinal Mucosa Physiology and Health

Any inflammatory process in the gut may result in pathological changes to the intestine (e.g. see FIG. 5). Furthermore, previous studies also showed that challenge with pathogens such as Clostridium perfringens or E. coli, even in the absence of fulminant enteritis produces an increased rate of exfoliation from the tip of the villi. In order to restore normal physiological function and health of the intestinal mucosa, there is a need for rapid regeneration of damaged villi.

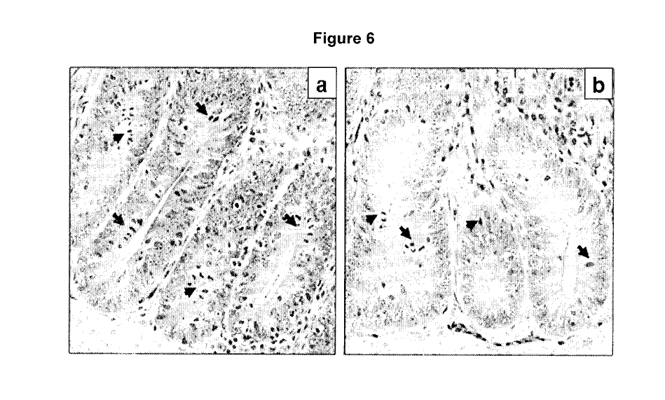

In direct relevance to the present investigation were observations that administration of an LCP extract of flaxseed in the diet appears to stimulate mitotic activity in the crypts of intestinal mucosa, as evidenced by overall larger numbers of mitotic figures (FIG. 6).

The present observations suggest that the broiler chickens fed diets supplemented with an LCP extract from flaxseed may have much higher potential for re-generation of damaged villi. These findings are of practical importance, for example, because in cases of enteritis, robust re-generation of the damaged intestinal mucosa would be a very desirable feature. It is important to stress that the continuous, active proliferation of the enterocytes at the crypt level is essential for the physiology of the entire villus, and any disruption of this process will inevitably lead to death of the villus.

### (h) General Observations Related to Health

The trials conducted thus far revealed several trends indicative of beneficial effects of an LCP extract from flaxseed on subject health. For example, the two following observations were made with regard to prominent health problems commonly occurring in fast growing broiler chickens in a commercial setting.

(i) Foot Pad Lesions:

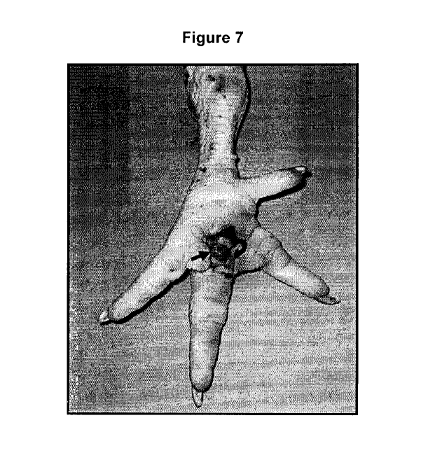

Foot pad lesions are a growing concern in the broiler chicken industry throughout the world. These lesions are characterized by progressive necrotic erosions of the soft tissue, accompanied by hemorrhages and inflammatory reactions. A mixture of inflammatory exudates, litter, and faecal material, usually further contaminate the open ulcerations (FIG. 7).

The incidence of foot pad lesions can be observed in about 20 to 80% of otherwise normal broiler chicken populations. Any foot pad lesions are a cause of distress, but in more severe cases these lesions are likely associated with severe pain and markedly decreased walking ability.

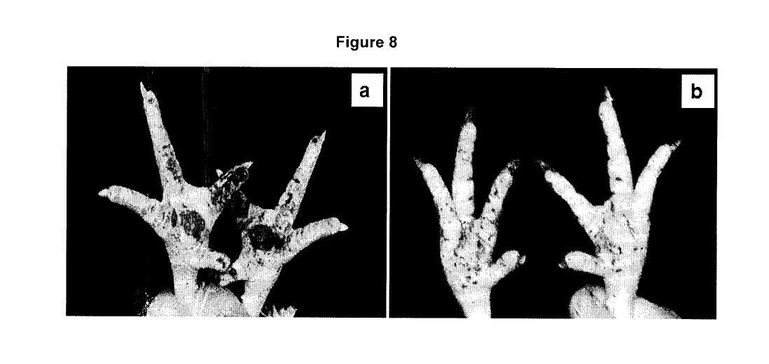

During the course of the trials conducted herein, it was observed that, in comparison to control broiler chickens fed a basal diet, the groups of broiler chickens fed diets supplemented with an LCP extract showed a considerably lower incidence of foot pad lesions (FIG. 8).

(ii) Coxo-Femoral Joint Lesions:

The coxo-femoral joint lesions and femoral head necrosis represent the most common condition associated with lameness in commercial broiler chicken flocks. Severe lesions in the femoral head are observed in 10 to 15% of commercial broiler chickens. Other studies have reported an incidence between 14% and 30% of birds showing severely impaired gait in different European countries (Sanotra, G. S. et al. World Poultry Science Journal, 2001, 57, 55-69; Sanotra, G. S. et al. Animal Welfare, 2003, 12, 677-683; Dawkins, M. S. et al. Nature, 2004, 427, 342-344; Knowles, T. et al. PloS ONE, 2008, 2, e1545).

The clinical aspect of lesions associated with coxo-femoral joint anatomy is rather dramatic (see Olkowski A A, et al. Avian Pathol; 2011 December; 40(6):639-50 PMID: 22107098). The affected broiler chickens are reluctant to walk, but they do not show any overt signs of leg deformity. When forced to move, these birds may attempt, for example, crawling with the support of their wings rather than walk, hence in the jargon of producers they are frequently called “wing walkers”.

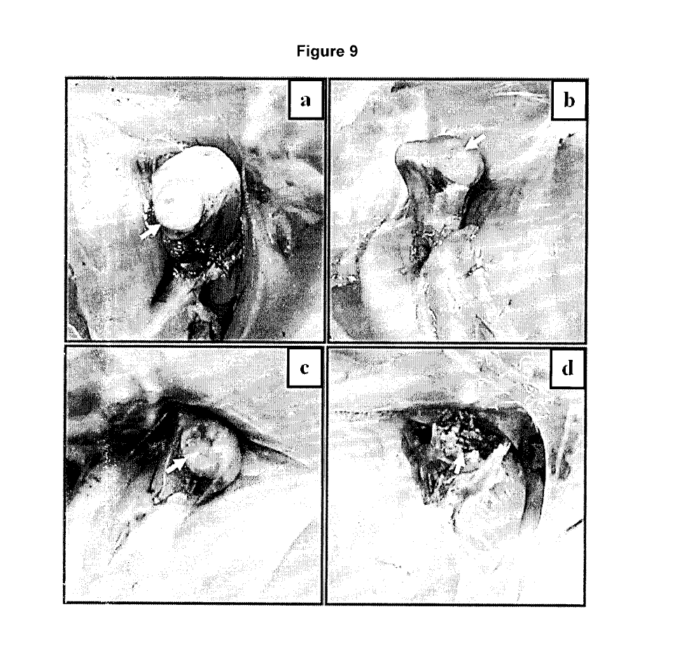

Observations made during the course of the trials indicate that in comparison to control broiler chickens fed a basal diet, those groups of broiler chickens fed diets supplemented with an LCP extract showed considerably reduced incidence of the coxo-femoral joint lesions, and in cases where such lesions did occur, their severity was reduced (FIG. 9).

In affected birds, routine disarticulation of coxo-femoral joint results in the cartilaginous epiphyseal plate separating readily from the bone metaphysic (FIG. 9b, white arrow), and as the degenerative process evolves, there is evident progressive loss of the sub-chondral bone and eburnation (FIG. 9c, white arrow). In more advanced stages, eventually the entire proximal part of the femur undergoes progressive necrosis (FIG. 9d, white arrow).

Examples of features indicative of femoral head degeneration seen on post mortem examination in broiler chickens fed a basal diet amended with an LCP extract would be similar to those demonstrated in FIG. 9b, whereas the lesions seen in broiler chickens from the control group fed a basal diet would be more typical to features demonstrated in FIGS. 9c and 9d.

### Example 3

### Evaluation of Anti Coccidial Activity

In this trial, a study focused on isolation of coccidian oocysts was conducted. Faecal matter samples collected from pens housing control and treated groups were screened for Coccidian oocysts according to standard techniques used in parasitological procedures for isolation of identification of parasite eggs and oocysts. This technique is based on the principle that parasite eggs and oocysts, being less dense than a flotation solution with a high specific gravity, will rise to the top of the media.

Eimeria spp. oocysts were found at low concentration of approximately 20 oocysts per gram of litter in samples collected from the pen housing birds fed control diet. There were no coccidia oocysts in the faecal samples collected from the pen housing broiler chickens fed a diet supplemented with an extract of LCP from flaxseed.

### Example 4

### Evaluation of the Effects of LCP on Coccidial Oocysts In Vitro

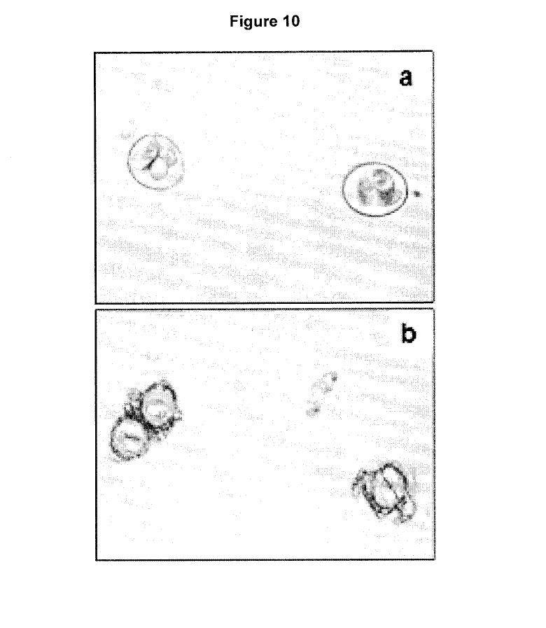

Further experiments were conducted using Coccidia oocysts freshly isolated from cattle, sheep, and chicken intestines according to standard operating procedures for the detection of parasite eggs and oocysts (Manual of Veterinary Parasitological Laboratory Techniques). The cultures of coccidial oocysts were incubated in medium containing an LCP extract from flaxseed for 24 hours, and the morphology of oocysts was evaluated. These in vitro assays showed that the LCP extract exerts a toxic effect on Eimeria oocysts. In the cultures treated with an LCP extract, Eimeria oocysts showed signs of severe injury as evidenced by blebbing of the oocyst shell. The lethality of the oocysts' injury was also evidenced by a halting of the progression of sporulation (FIG. 10).

During preparation of faecal material for the isolation of coccidial oocysts, it was noticed that the faecal fraction dissolved in water. This faecal solution was able to sediment on gravity and contained large structures filled with some spores. It was concluded that these structures were definitively not protozoa, and were likely (unidentified) fungal spores.

A small culture of these structures was prepared and allocated into two 1 mL aliquots. The samples were then subjected to an in vitro test. One sample was mixed with 20 μL of DMSO, whereas the other was mixed with 20 μL of DMSO containing an LCP extract from flaxseed. The samples were then incubated at room temperature (RT) for one hour. Following incubation, 20 μL of methylene blue solution was added to each culture, and incubated for 30 min. Following this, the viability of these spores was assessed under the microscope using the dye exclusion test. This test is based on the principle that a viable cell can exclude stain and therefore remains unstained in the assay, whereas those cells that are dead cannot exclude stain and therefore are stained in blue.

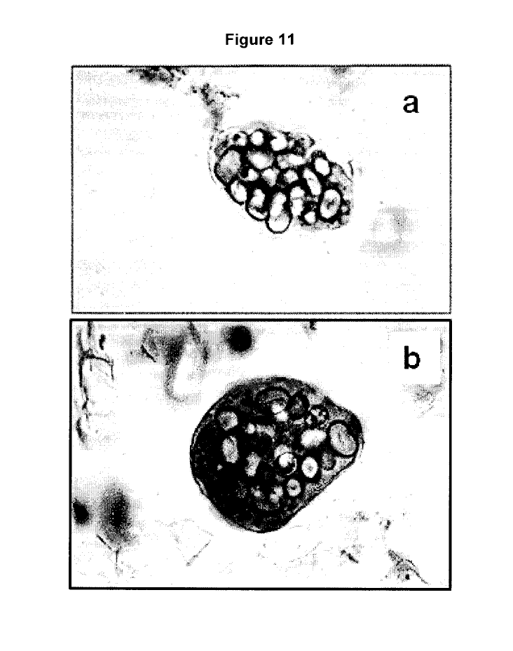

This in vitro test revealed that an LCP extract of flaxseed may be effective in controlling the proliferation of fungal spores (FIG. 11).

Spores from control cultures (a) which were incubated only with DMSO remained unstained, and thus were viable. On the other hand, spores incubated with DMSO containing an LCP extract from flaxseed (b) were stained blue, which indicated that these spores were not viable.

The observation from the in vitro test using yet to be identified formations containing spores indicates that an LCP extract from flaxseed may have fungicidal properties.

### Example 5

### Evaluation of Fungicidal Activity

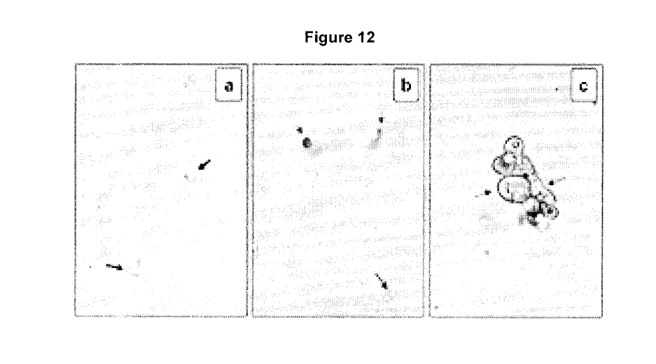

The evaluation of putative antimicrobial properties of an LCP extract of flaxseed was performed in vitro using C. albicans cells as a model organism. Briefly: samples of 2×10⁶ C. albicans cells suspended in 0.6 mL of Hank's Balanced Salt Solution (HBSS) were mixed in test tubes with an LCP extract of flaxseed dissolved in DMSO (concentration approximately 5 mg/mL assay) or DMSO. The tubes were capped and incubated for 12 hours at 37° C. Then, 0.25 mL of 0.2 mM methylene blue was added and incubation was allowed to continue for a further 30 min. Following this, the samples were assessed for viability and morphological changes of C. albicans cells. This study showed that an LCP extract of flaxseed is an effective agent capable of inflicting severe injury to the cellular membrane and killing C. albicans cells (FIG. 12).

### Example 6

### Microbiological Evaluation of Intestinal Content

During the 6^{th} week of the trial, sections of the small intestines were collected and the content was subjected to microbiological evaluation. The intestinal contents were cultured using two plates of Columbia blood agar and one of MacConkey agar (BBL, Beckton Dickinson). One blood agar plate was incubated at 37° C. in an atmosphere of 5% CO₂ for 24 hours, at which time subcultures of the bacterial colonies were performed for further identification and the plates were re-incubated for another 24 hours to detect possible slow growers. The other blood agar plate was incubated anaerobically at 37° C. for 48 hours. The MacConkey agar was incubated for 24 hours aerobically at 37° C. Bacteria identification was performed by standard methods.

No anaerobic bacteria were cultured from diluted or direct samples. Diluted sample showed low overall counts of aerobic bacteria, and some aerobic species were cultured only from direct samples.

There was a general trend showing lower E. coli numbers in specimens from broiler chickens treated with an LCP extract from flaxseed.

### Example 7

### Antiviral Activity of Flax Peptide Extract

During the course of the feeding trial three cases of mortality associated with severe pathological changes were noted in the liver. Following histological examination, the condition was diagnosed as inclusion body hepatitis.

Inclusion body hepatitis (IBH) is a viral disease commonly observed in broiler flocks, with mortality rate as high as 10% or even more in some instances.

In the context of the present study, of particular interest is the frequency of distribution of IBH cases. Notably, 2 out of 50 (4%) chickens allocated as the control group (fed basal diet) developed IBH, whereas in the groups fed diets supplemented with CLP only 1 out of 100 (1%) broilers showed IBH. Thus, the incidence of IBH was 4 fold higher among chickens fed basal diet in comparison to chickens fed diet supplemented with CLP.

Although limited in scope, based on the findings from the present studies, it is likely that CLPs have antiviral properties.

While the present disclosure has been described with reference to what are presently considered to be the preferred examples, it is to be understood that the disclosure is not limited to the disclosed examples. To the contrary, the disclosure is intended to cover various modifications and equivalent arrangements included within the spirit and scope of the appended claims.

TABLE 1
Representative body weights (g) for broiler chickens fed basal
diet (control group) and broiler chickens exposed to basal diet
amended with an LCP extract from flaxseed (treatment group).

| | Average Body Weight (grams) | | |
|---|---|---|---|
| | Day 7 | Day 14 | Day 21 |
| Control Group | 279.0 | 575.9 | 963.7 |
| Treatment Group | 281.6 | 580.8 | 966.3 |

The birds were weighed in 2 groups of 5 in each period, with a total n=10 per group.

TABLE 2
Incidence of morbidity and mortality among broiler chickens fed basal
diet (control group) and broiler chickens exposed to basal diet amended
with an LCP extract from flaxseed (treatment group).

| | Morbidity | Mortality |
|---|---|---|
| Control Group | 2/50 | 2/50 |
| | (4%) | (4%) |
| Treatment Group | 2/100 | 1/100 |
| | (2%) | (1%) |

TABLE 3
Incidence of enteritis observed during the first four weeks
of exposure among broiler chickens fed basal diet (control
group) and broiler chickens fed the basal diet amended with
an LCP extract from flaxseed (treatment group).

Cases of Enteritis Positive/
Number of Sampled

| | Day 7 | Day 14 | Day 21 | Day 28 |
|---|---|---|---|---|
| Control Group | 6/10 | 5/10 | 5/8 | 3/5 |
| | (60%) | (50%) | (62.5%) | (60%) |
| Treatment Group | 0/10 | 2/10 | 3/8 | 2/12 |
| | (0%) | (20%) | (37.5%) | (16.7%) |

1. A method for improving health of a subject comprising administering to the subject an effective amount of an LCP extract from flaxseed.

2. The method of claim 1, wherein improving health comprises one of more of: increasing growth rates and/or body weight, decreasing morbidity, decreasing mortality, decreasing enteritis, decreasing fungal growth, modulating gastro-intestinal tract micro-organisms, enhancing cell-cycle activity and inhibiting infiltration of inflammatory cells into the intestinal mucosa.

3. The method of claim 1, wherein the LCP extract is obtained using a method comprising:
(a) combining flaxseed oil with an adsorbent that binds the hydrophobic peptides from the oil, followed by separation of the adsorbent and extraction of the hydrophobic peptides from the adsorbent using a suitable solvent; or
(b) extracting flaxseeds, or flaxseed oil, with a suitable solvent and separating the LCPs from the polar solvent using silica gel solid phase extraction using a suitable solvent.

4. The method of claim 3, wherein the suitable solvent is ethyl acetate, 10% methanol in chloroform or another solvent or mixture of solvents having a polarity similar to these solvents.

5. The method of claim 1, wherein the LCP extract from flaxseed oil comprises, consists essentially of or consists of LCP-1, LCP-2, LCP-3, LCP-4, LCP-5, LCP-6, LCP-7, LCP-8 and LCP-9-, in amounts that correspond to their amounts found in the flaxseed.

6. The method of claim 5, wherein the LCP extract from flaxseed comprises, consists essentially of or consists of LCP-1, LCP-3, LCP-4, LCP-5, LCP-6 and LCP-7.

7. The method of claim 1, wherein the LCP extract from flaxseed comprises, consists essentially of or consists of:
(a) about 15% to about 30% of each of LCP-1, LCP-3 and LCP-5;
(b) about 10% to about 20% of LCP-7; and
(c) about 2% to about 15% of each of LCP-4 and LCP-6.

8. The method of claim 1, the LCP extract from flaxseed comprises greater than about 75, 80, 85, 90 or 95% of LCPs from flaxseed.

9. The method of claim 1, wherein the LCP extract from flaxseed is administered by feeding the subject a diet comprising the LCP extract from flaxseed.

10. The method of claim 1, wherein the increasing, decreasing or inhibiting by the LCP extract from flaxseed is any increase, decrease or inhibition compared to a control.

11. The method of claim 10, wherein the control is a control diet that is identical to the diet comprising the LCP extract from flaxseed, with the exception that it does not comprise the LCP extract from flaxseed.

12. The method of claim 1, wherein the enhancement in cell-cycle activity is in the bursa, intestinal mucosa and/or in the enterocytes.

13. The method of claim 1, wherein the subject is a human or an animal selected from livestock and pets.

14. The method of claim 13, wherein the subject is selected from a bovine, feline, equine, porcine, canine, aquatic, avian, caprine, ovine and murine animal.

15. The method of claim 14, wherein the bovine animal is selected from beef cattle and dairy cows; the porcine animal is a hog; the ovine animal is a sheep; the avian animal is a poultry animal; the caprine animal is a goat; the aquatic animal is selected from fish, crayfish and crustaceans; the canine animal is a domesticated dog;
and the feline animal is a domesticated cat.

16. The method of claim 15, wherein the poultry animal is selected from chickens, turkeys and ducks.

17. A feed or food stuff, a food supplement composition, a nutraceutical, a functional food composition, a natural product composition or a pharmaceutical composition, comprising an LCP extract from flaxseed.

18. The feed or food stuff, food supplement composition, nutraceutical, functional food composition, natural product composition or pharmaceutical composition of claim 17, wherein the LCP extract from flaxseed oil comprises, consists essentially of or consists of LCP-1, LCP-2, LCP-3, LCP-4, LCP-5, LCP-6, LCP-7, LCP-8 and LCP-9, in amounts that correspond to their amounts found in the flaxseed.

19. The feed or food stuff, food supplement composition, nutraceutical, functional food composition, natural product composition or pharmaceutical composition of claim 17, formulated for administration as a powder, a bulb, a capsule, a pill, a tablet, a liquid, a suspension, a gel, a cream or an ointment.

20. The feed or food stuff, food supplement composition, nutraceutical, functional food composition, natural product composition or pharmaceutical composition of claim 17, wherein the LCP extract from flaxseed is obtained using a method comprising:
(a) combining flaxseed oil with an adsorbent that binds the hydrophobic peptides from the oil, followed by separation of the adsorbent and extraction of the hydrophobic peptides from the adsorbent using a suitable solvent; or
(b) extracting flaxseeds, or flaxseed oil, with a suitable solvent and separating the LCPs from the polar solvent using silica gel solid phase extraction using a suitable solvent.
